(19) United States
(12) Patent Application Publication
(10) Pub. No.: US 2024/0408542 A1
(43) Pub. Date: Dec. 12, 2024

(54) AIR PURIFIER WITH FUNCTION OF INACTIVATING COLLECTED PATHOGENS IN FILTER PORTION

(71) Applicant: KOREA INSTITUTE OF MATERIALS SCIENCE, Changwon-si (KR)

(72) Inventors: Seung-hoon LEE, Changwon-si (KR); Do-geun KIM, Changwon-si (KR); Joo-young PARK, Changwon-si (KR); Eun-yeon BYEON, Changwon-si (KR); Sung-hoon JUNG, Changwon-si (KR)

(21) Appl. No.: 18/738,177

(22) Filed: Jun. 10, 2024

(30) Foreign Application Priority Data
Jun. 9, 2023 (KR) 10-2023-0074061

(51) Int. Cl. B01D 53/86; A61L 2/14; A61L 2/20; A61L 2/26; B01D 46/00; B01D 46/80; C01B 13/10

(52) U.S. Cl. CPC B01D 53/8675; A61L 2/14; A61L 2/202; A61L 2/26; B01D 46/0028; B01D 46/0045; B01D 46/80; C01B 13/10; A61L 2202/11; A61L 2202/13; A61L 2202/15; A61L 2209/14; A61L 2209/212; B01D 2257/106; B01D 2257/91; B01D 2258/06; B01D 2273/30; B01D 2279/65

(57) ABSTRACT

The present application provides an air purifier with a function of inactivating collected pathogens in a filter portion. More specifically, the present application provides an air purifier with a function of inactivating collected pathogens in a filter portion, the air purifier being capable of efficiently inactivating collected pathogens in the filter portion while protecting a pathogen collection filter portion and significantly reducing ozone to be treated by an ozone catalyst portion by optimizing operation of a plasma filter portion, and an air purifying method using the same.

### CROSS REFERENCE TO RELATED APPLICATION

This application claims the benefit of Korean Patent Application No. 10-2023-0074061, filed on, Jun. 9, 2023, which is hereby incorporated by reference in its entirety into this application.

### BACKGROUND OF THE INVENTION

### 1. Technical Field

The present application relates to an air purifier with a function of inactivating collected pathogens in a filter portion, and more particularly, to an air purifier with a function of inactivating collected pathogens in a filter portion, the air purifier being capable of efficiently inactivating collected pathogens in the filter portion while protecting a polymer fiber filter portion and significantly reducing ozone to be treated by an ozone catalyst portion by optimizing operation of a plasma filter portion, and an air purifying method using the same.

### 2. Description of the Related Art

A filter fan, a common component of an air purifier, may not biologically inactivate bacteria or virus particles (hereinafter, referred to as pathogens) collected in a filter. Therefore, technologies such as copper coating and ultraviolet irradiation have been applied to inactivate pathogens attached to the surface of the filter.

However, in a case of applying the copper coating, when a coating surface is contaminated with dust or the like, an effect of inactivating collected pathogens by copper ions released from the coating decreases. In a case of applying the ultraviolet irradiation, it is difficult to inactivate pathogens collected at positions where light may not reach, and damage to polymer fibers of the filter occurs due to long-term exposure to ultraviolet rays.

An alternative that may solve the above problem is a low-temperature plasma method. Air plasma generated at atmospheric pressure and low temperature may generate a large amount of ozone gas with high oxidizing power. A plasma device that is manufactured in the form of a filter supplies ozone gas to pathogens collected in all regions including the surface of the filter and the interior of the filter to inactivate the pathogens through an oxidation reaction. However, ozone released after the oxidation of the pathogens to the outside of the air purifier may cause damage to the respiratory tract when inhaled by a person.

An integrated component, in which plasma, a polymer filter, and a catalyst are combined, has been proposed to solve the above problem. The integrated component may inactivate collected pathogens in the filter by continuously exposing the pathogens to ozone, and remove the released ozone with a catalyst component. However, continuous ozone generation increases the burden on the catalyst component to remove ozone. Therefore, except in special situations where continuous removal of collected pathogens is necessary, it is practical to inactivate collected pathogens in a polymer filter portion through non-continuous plasma operation.

However, in a case of the non-continuous plasma operation as described above, air purification efficiency such as pathogen inactivation efficiency may deteriorate.

In addition, as the non-continuous plasma operation is applied to normal operation and the area of a plasma filter increases, an air purifier modified from the integrated component type has been required. Furthermore, while increasing the area of the plasma filter is straightforward in terms of cost and mechanism, increasing the area of an ozone removal catalyst is difficult.

Korean Patent Laid-Open Publication No. 10-2021-0059098 describes a portable plasma generation device as a background technology of the present application.

### SUMMARY OF THE INVENTION

An object of the present application is to provide an air purifier with a function of inactivating collected pathogens in a filter portion, which may efficiently inactivate the collected pathogens in the filter portion while protecting a polymer fiber filter portion and significantly reducing ozone to be treated in an ozone catalyst portion.

Another object of the present application is to provide an air purifier with a function of inactivating collected pathogens in a filter portion, which may efficiently inactivate the collected pathogens in the filter portion in a case of being operated in a manner of removing the collected pathogens in the filter portion through non-continuous operation of a plasma filter portion.

Another object of the present application is to provide a method capable of efficiently inactivating collected pathogens in a filter portion in a case of operating an air purifier in a manner of removing the collected pathogens in the filter portion through non-continuous operation of a plasma filter portion.

Other objects and advantages of the present invention will become more apparent from the following detailed description, claims, and drawings.

According to an aspect, an air purifier with a function of inactivating collected pathogens in a filter portion includes: a tubular body portion having an air intake port; a plasma filter portion provided behind the air intake port; a pathogen collection filter portion provided on at least one side of the plasma filter portion; and a bypass passage guiding an air flow and including an ozone decomposition portion, the air flow containing ozone generated during operation of the plasma filter portion and passing through the pathogen collection filter portion.

The bypass passage may include a fan guiding the air flow into the bypass passage and discharging air passing through the ozone decomposition portion indoors, the air flow containing ozone generated during operation of the plasma filter portion and passing through the pathogen collection filter portion.

The plasma filter portion may include a plasma generation module including a first ground electrode, a high voltage electrode, and a ceramic layer formed of a porous ceramic dielectric between the first ground electrode and the high voltage electrode, and the first ground electrode and the high voltage electrode are formed of a grid-shaped or porous metal.

The air purifier may include: the tubular body portion having the air intake port; the plasma filter portion provided behind the air intake port; the pathogen collection filter portion provided on at least one side of the plasma filter portion; a first fan guiding air passing through the pathogen collection filter portion to a first passage when the plasma filter portion is not in operation; a first air discharge port discharging air passing through the first passage indoors; a second fan guiding air passing through the pathogen collection filter portion to a second passage as the bypass passage when the plasma filter portion is in operation to inactivate the pathogens collected in the pathogen collection filter portion; a second air discharge port discharging air passing through the second passage indoors; and the ozone decomposition portion included in the second passage and decomposing ozone generated in the plasma filter portion before the air is discharged through the second air discharge port.

The first fan may be operated at 1000 to 1500 CMH when the plasma filter portion is not in operation.

The second fan may be operated at 10 to 200 CMH when the plasma filter portion is in operation.

The pathogen collection filter portion may include a high efficiency particulate air (HEPA) filter.

The ozone decomposition portion may include an ozone decomposition catalyst.

According to another aspect, an air purifying method using the air purifier described in the present application includes: a pathogen inactivation mode step of guiding air containing ozone generated during operation of the plasma filter portion and passing through the pathogen collection filter portion into the bypass passage, and discharging air passing through the ozone decomposition portion indoors.

According to still another aspect, an air purifying method using the air purifier described in the present application includes: a normal operation mode step of guiding the air passing through the pathogen collection filter portion to the first passage by operation of the first fan when the plasma filter portion is not in operation and discharging the air passing through the first passage e indoors through the first air discharge port; and a pathogen inactivation mode step of guiding the air passing through the pathogen collection filter portion to the second passage by operation of the second fan when the plasma filter portion is in operation and discharging the air passing through the second passage and passing through the ozone decomposition portion indoors through the second air discharge port.

### BRIEF DESCRIPTION OF THE DRAWINGS

FIG. 1 is a schematic cross-sectional view of an air purifier, which illustrates an air flow A1 in a normal operation mode according to an exemplary embodiment of the present application;

FIG. 2 is a schematic cross-sectional view of the air purifier, which illustrates an air flow A2 in a pathogen inactivation mode according to the exemplary embodiment of the present application;

FIG. 3 is a diagram schematically illustrating the air flow and an operation sequence in the normal operation mode according to the exemplary embodiment of the present application; and

FIG. 4 is a diagram schematically illustrating the air flow and an operation sequence in the pathogen inactivation mode according to the exemplary embodiment of the present application.

### DESCRIPTION OF EXEMPLARY EMBODIMENTS

The above-mentioned objects and means of the present invention, and effects thereof will become more obvious from the following detailed description associated with the accompanying drawings. Therefore, those skilled in the art to which the present invention pertains may easily practice a technical idea of the present invention. Further, in describing the present invention, when a detailed description of well-known technology relating to the present invention may unnecessarily make unclear the spirit of the present invention, a detailed description thereof will be omitted.

Terms used in the present specification are for describing exemplary embodiments rather than limiting the present invention. In the present specification, a singular form may include a plural form as needed unless explicitly stated otherwise. The term “include”, “comprise”, “provide”, or “have” used in the present specification do not exclude the existence or addition of one or more other components other than the mentioned components.

In the present specification, terms such as “or” and “at least one” may represent one of words listed together, or a combination of two or more thereof. For example, “A or B”, “at least one of A or B” may include only A or B, or both A and B.

In the present specification, presented information such as cited characteristics, variables, or values may not precisely match in descriptions following phrases such as “for example”, and modes according to various exemplary embodiments of the present invention should not be limited by effects such as modifications including limits of tolerances, measurement errors, and measurement accuracy, and other commonly known factors.

In the present specification, it is to be understood that when one constituent element is described as being “connected to” or “coupled to” another constituent element, it may be connected directly to or coupled directly to another constituent element or be connected to or coupled to another constituent element, having the other constituent element intervening therebetween. On the other hand, it is to be understood that when one constituent element is referred to as being “connected directly to” or “coupled directly to” another constituent element, it may be connected to or coupled to another constituent element without the other constituent element intervening therebetween.

In the present specification, it is to be understood that when one constituent element is described as being “on” or “in contact with” another constituent element, one constituent element may be in direct contact with or be connected directly to another constituent element, or the other constituent element may exist between one constituent element and another constituent element. On the other hand, when one constituent element is described as being “directly on” or “in direct contact with” another constituent element, it may be understood that the other constituent element does not exist between one constituent element and another constituent element. Other expressions describing a relationship between constituent elements, for example, “between” and “directly between” may be similarly interpreted.

Terms “first”, “second”, and the like, may be used to describe various constituent elements, but the constituent elements are not to be construed as being limited by these terms. In addition, the above terms should not be interpreted as limiting the order of each constituent element but may be used for the purpose of distinguishing one constituent element from another constituent element. For example, a “first” constituent element may be named a “second” constituent element and the “second” constituent element may also be similarly named the “first” constituent element.

Unless defined otherwise, all terms used in the present specification have the same meaning as meanings commonly understood by those skilled in the art to which the present invention pertains. In addition, terms defined in generally used dictionaries are not ideally or excessively interpreted unless they are specifically defined clearly.

Hereinafter, an exemplary embodiment of the present invention will be described in detail with reference to the accompanying drawings.

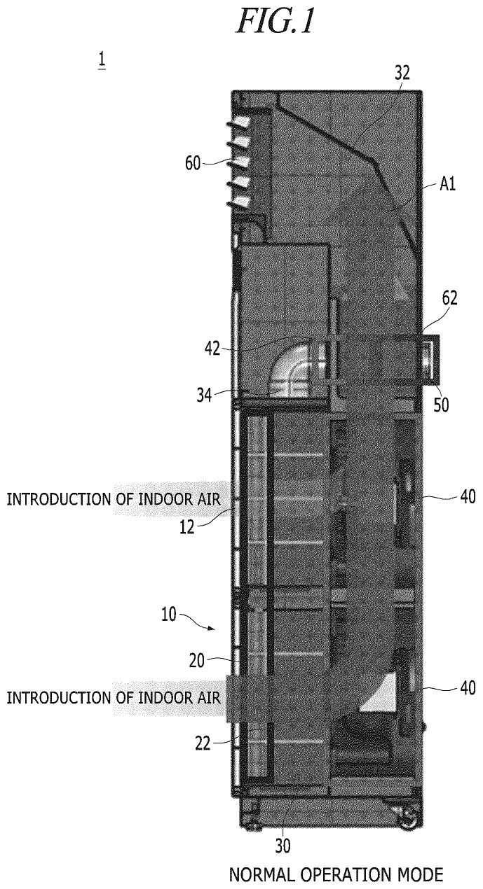

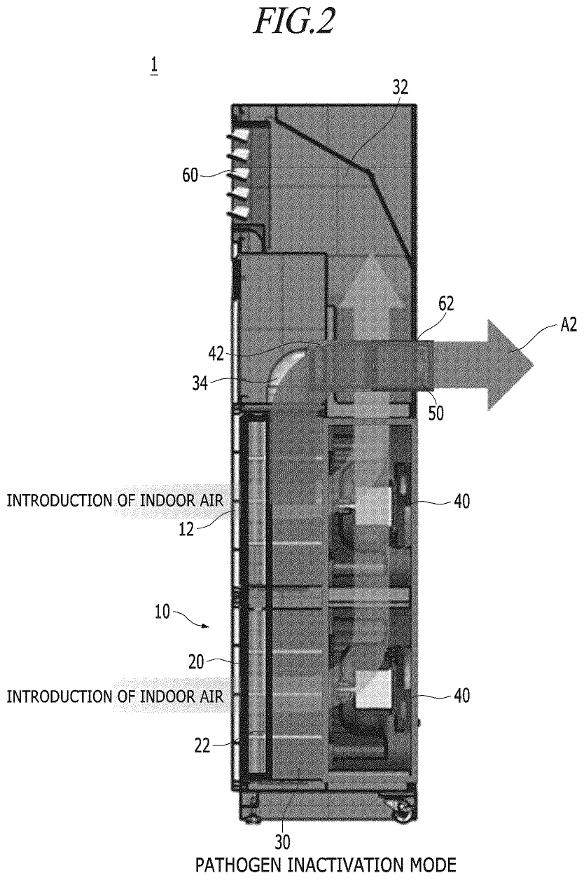

FIG. 1 is a schematic cross-sectional view of an air purifier, which illustrates an air flow A1 in a normal operation mode according to an exemplary embodiment of the present application, and FIG. 2 is a schematic cross-sectional view of the air purifier, which illustrates an air flow A2 in a pathogen inactivation mode according to the exemplary embodiment of the present application.

Referring to FIGS. 1 and 2, an air purifier 1 with a function of inactivating collected pathogens in a filter portion according to the present application may include a tubular body portion 10 having an air intake port 12, a plasma filter portion provided behind the air intake port 12, a pathogen collection filter portion 22 provided on at least one side of the plasma filter portion 20, and a bypass passage guiding the air flow A2 and including an ozone decomposition portion 50, the air flow A2 containing ozone generated during operation of the plasma filter portion 20 and passing through the pathogen collection filter portion 22.

The bypass passage may include a fan guiding the air flow A2 to the bypass passage and discharging air passing through the ozone decomposition portion 50 indoors, the air flow A2 containing ozone generated during operation of the plasma filter portion 20 and passing through the pathogen collection filter portion 22, but the bypass passage is not limited thereto.

Active species such as ozone are generated during operation of the plasma filter portion 20 and inactivate the pathogens collected in the plasma filter portion 20 and the pathogen collection filter portion 22. At this time, with the above-described structure, the air flow A2 containing ozone and passing through the pathogen collection filter portion 22 may be guided by the bypass passage and/or the fan, and ozone may be decomposed by the ozone decomposition portion 50 included in the bypass passage.

Therefore, only the air flow A2 containing ozone may be guided to the bypass passage including the ozone decomposition portion 50 to remove ozone by inactivating the pathogens collected in the plasma filter portion 20 and the pathogen collection filter portion 22 for a certain period of time by operating the plasma filter portion 20 at a certain cycle, thereby preventing overload of the ozone decomposition portion 50. In addition, it is possible to minimize damage to the pathogen collection filter portion 22 implemented by a polymer fiber filter due to continuous operation of the plasma filter portion 20.

The plasma filter portion 20 may include a plasma generation module including a first ground electrode, a high voltage electrode, and a ceramic layer formed of a porous ceramic dielectric between the first ground electrode and the high voltage electrode, and the first ground electrode and the high voltage electrode may be formed of a grid-shaped or porous metal. However, the plasma filter portion 20 is not limited thereto.

The plasma filter portion 20 according to the present application may be low-temperature plasma operated with a low-frequency power supply, but is not limited thereto. With the above-described configuration, it is possible to ensure safety for human bodies and improve reliability, safety, and durability of the plasma filter portion 20 through protection of the dielectric and/or polymer fiber filter portion.

There are no particular restrictions on the type and position of the pathogen collection filter portion 22 as long as the pathogen collection filter portion 22 may collect pathogens floating in the air sucked through the air intake port 12. The pathogens collected in the pathogen collection filter portion 22 may be inactivated by the active species such as ozone generated in the plasma filter portion 20. The pathogens may include bacteria, fungi, and viruses, but are not limited thereto. The pathogen collection filter portion 22 may include a high efficiency particulate air (HEPA) filter, but is not limited thereto.

The ozone decomposition portion 50 has no particular limitations as long as the ozone decomposition portion 50 may decompose ozone. The ozone decomposition portion 50 may include a known ozone decomposition catalyst or an ozone decomposition device. For example, the ozone decomposition portion 50 may be a filter containing an ozone removal catalyst or an ozone decomposition catalyst in which a carbon composite, activated carbon particles, manganese dioxide, copper oxide and/or an active material is supported on a support containing Pd and/or Pt, but is not limited thereto.

Further, an installation position of the ozone decomposition portion 50 is not particularly limited as long as the ozone decomposition portion 50 may decompose ozone before air is discharged from the bypass passage.

Referring to FIGS. 1 and 2, the air purifier 1 may include the tubular body portion 10 having the air intake port 12, the plasma filter portion 20 provided behind the air intake port 12, the pathogen collection filter portion 22 provided on at least one side of the plasma filter portion 20, a first fan 40 guiding air passing through the pathogen collection filter portion 22 to a first passage 32 when the plasma filter portion 20 is not in operation, a first air discharge port 60 discharging air passing through the first passage 32 indoors, a second fan 42 guiding air passing through the pathogen collection filter portion 22 to a second passage 34 as the bypass passage when the plasma filter portion 20 is in operation to inactivate the pathogens collected in the pathogen collection filter portion 22, a second air discharge port 62 discharging the air passing through the second passage 34 indoors, and the ozone decomposition portion 50 included in the second passage 34 and decomposing ozone generated in the plasma filter portion 20 before the air is discharged through the second air discharge port 62.

The first fan 40 may be operated at a wind volume of 1000 to 1500 CMH when the plasma filter portion 20 is not in operation, but is not limited thereto.

The second fan 42 may be operated at a wind volume of 10 to 200 CMH when the plasma filter portion 20 is in operation, but is not limited thereto. With the above-described configuration, an ozone concentration in the pathogen collection filter portion 22 may be maintained high even with low power consumption of the plasma filter portion 20, and the amount of ozone that needs to be substantially treated by the catalyst may be significantly reduced.

For efficient inactivation of pathogens, the plasma filter portion 20 may be operated in such a way that the ozone concentration in the pathogen collection filter portion 22 is maintained at a level of 0.5 to 5 ppm, but is not limited thereto.

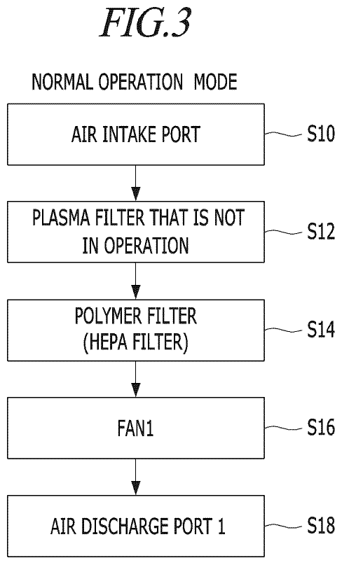

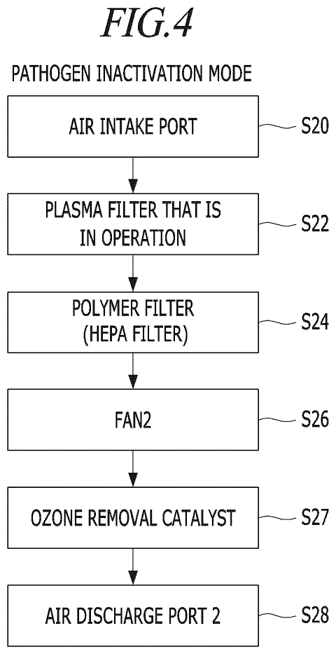

FIG. 3 is a diagram schematically illustrating the air flow and an operation sequence in the normal operation mode according to the exemplary embodiment of the present application, and FIG. 4 is a diagram schematically illustrating the air flow and an operation sequence in the pathogen inactivation mode according to the exemplary embodiment of the present application.

Referring to FIGS. 2 and 4, an air purifying method using the air purifier with a function of inactivating collected pathogens in a filter portion described herein according to another aspect may include a pathogen inactivation mode step of guiding air containing ozone generated during operation of the plasma filter portion 20 and passing through the pathogen collection filter portion 22 to the bypass passage, and discharging air passing through the ozone decomposition portion 50 indoors.

With the above-described configuration, the active species such as ozone are generated during operation of the plasma filter portion 20 and inactivate the pathogens collected in the plasma filter portion 20 and the pathogen collection filter portion 22. At this time, with the above-described configuration, the air flow A2 containing ozone and passing through the pathogen collection filter portion 22 may be guided by the second passage 34 as the bypass passage, and ozone may be decomposed by the ozone decomposition portion 50 included in the second passage 34 as the bypass passage. Therefore, only the air flow containing ozone may be guided to the second passage 34 as the bypass passage including the ozone decomposition portion 50 to remove ozone by inactivating the pathogens collected in the plasma filter portion 20 and the pathogen collection filter portion 22 for a certain period of time by operating the plasma filter portion 20 at a certain cycle, thereby preventing overload of the ozone decomposition portion 50. In addition, it is possible to minimize damage to the pathogen collection filter portion 22 implemented by a polymer fiber filter due to continuous operation of the plasma filter portion 20.

Referring to FIGS. 1 to 4, an air purifying method using the air purifier with a function of inactivating collected pathogens in a filter portion described herein according to still another aspect may include a normal operation mode step and a pathogen inactivation mode step.

The air flow A1 in the normal operation mode step will be described in detail with reference to FIGS. 1 and 3.

Contaminated air passing through the air intake port 12 (S10) passes through the plasma filter portion 20 that is not in operation (S12). Next, pathogens contained in the air are collected in the polymer fiber filter as the pathogen collection filter portion 22 (S14), and the purified air passing through the pathogen collection filter portion 22 is guided to the first passage 32 by operation of the first fan 40 provided in a space portion 30 (S16), and the air passing through the first passage 32 is discharged indoors through the first air discharge port 60 (S18).

The air flow A2 in the pathogen inactivation mode step will be described in detail with reference to FIGS. 2 and 4.

Contaminated air passing through the air intake port 12 (S20) passes through the plasma filter portion 20 that is in operation (S22), and pathogens contained in the air are inactivated. In addition, pathogens collected in the polymer fiber filter as the pathogen collection filter portion 22 are also inactivated (S24). The air containing ozone or the like and passing through the pathogen collection filter portion 22 is guided to the second passage 34 as the bypass passage by operation of the second fan 42 (S26), and ozone is removed by the ozone decomposition portion 50 included in the second passage 34 (S27). The air from which ozone is removed is discharged indoors through the second air discharge port 62 (S28).

According to an exemplary embodiment, the air purifier with a function of inactivating collected pathogens in a filter portion according to the present application may include the passage that may guide a different air flow between when the plasma filter portion is in operation and when the plasma filter portion is not in operation, thereby efficiently inactivating the collected pathogens in the filter portion while significantly reducing ozone to be treated in the ozone catalyst portion.

According to an exemplary embodiment, the air purifier with a function of inactivating collected pathogens in a filter portion according to the present application may include the passage that may guide a different air flow between when the plasma filter portion is in operation and when the plasma filter portion is not in operation, thereby efficiently inactivating the collected pathogens in the filter portion while protecting the polymer fiber filter portion in which the pathogens are collected.

According to an exemplary embodiment, the air purifying method according to the present application may guide a different air flow between when the plasma filter portion is in operation and when the plasma filter portion is not in operation in a case of operating the air purifier in a manner of removing collected pathogens in the filter portion through non-continuous operation of the plasma filter portion, thereby efficiently inactivating the collected pathogens in the filter portion while protecting the polymer fiber filter portion and significantly reducing ozone to be treated in the ozone catalyst portion.

While specific exemplary embodiments have been described in the detailed description of the present invention, various modifications may be made without departing from the scope of the present invention. Therefore, the scope of the present invention is defined not by the described exemplary embodiments but by the appended claims as well as equivalents thereto.

What is claimed is:

1. An air purifier with a function of inactivating collected pathogens in a filter portion, the air purifier comprising:
a tubular body portion having an air intake port;
a plasma filter portion provided behind the air intake port;
a pathogen collection filter portion provided on at least one side of the plasma filter portion; and
a bypass passage guiding an air flow and including an ozone decomposition portion, the air flow containing ozone generated during operation of the plasma filter portion and passing through the pathogen collection filter portion.

2. The air purifier of claim 1, wherein the bypass passage includes a fan guiding the air flow to the bypass passage and discharging air passing through the ozone decomposition portion indoors, the air flow containing ozone generated during operation of the plasma filter portion and passing through the pathogen collection filter portion.

3. The air purifier of claim 1, wherein the plasma filter portion includes a plasma generation module including a first ground electrode, a high voltage electrode, and a ceramic layer formed of a porous ceramic dielectric between the first ground electrode and the high voltage electrode, and
the first ground electrode and the high voltage electrode are formed of a grid-shaped or porous metal.

4. The air purifier of claim 1, wherein the air purifier comprises:
the tubular body portion having the air intake port;
the plasma filter portion provided behind the air intake port;
the pathogen collection filter portion provided on at least one side of the plasma filter portion;
a first fan guiding air passing through the pathogen collection filter portion to a first passage when the plasma filter portion is not in operation;
a first air discharge port discharging air passing through the first passage indoors;
a second fan guiding air passing through the pathogen collection filter portion to a second passage as the bypass passage when the plasma filter portion is in operation to inactivate the pathogens collected in the pathogen collection filter portion;
a second air discharge port discharging air passing through the second passage indoors; and
the ozone decomposition portion included in the second passage and decomposing ozone generated in the plasma filter portion before the air is discharged through the second air discharge port.

5. The air purifier of claim 4, wherein the first fan is operated at 1000 to 1500 CMH when the plasma filter portion is not in operation.

6. The air purifier of claim 4, wherein the second fan is operated at 10 to 200 CMH when the plasma filter portion is in operation.

7. The air purifier of claim 1, wherein the pathogen collection filter portion includes a high efficiency particulate air (HEPA) filter.

8. The air purifier of claim 1, wherein the ozone decomposition portion includes an ozone decomposition catalyst.

9. An air purifying method using the air purifier of claim 1, the air purifying method comprising:
a pathogen inactivation mode step of guiding air containing ozone generated during operation of the plasma filter portion and passing through the pathogen collection filter portion to the bypass passage, and discharging air passing through the ozone decomposition portion indoors.

10. An air purifying method using the air purifier of claim 4, the air purifying method comprising:
a normal operation mode step of guiding the air passing through the pathogen collection filter portion to the first passage by operation of the first fan when the plasma filter portion is not in operation and discharging the air passing through the first passage indoors through the first air discharge port; and
a pathogen inactivation mode step of guiding the air passing through the pathogen collection filter portion to the second passage by operation of the second fan when the plasma filter portion is in operation and discharging the air passing through the second passage and passing through the ozone decomposition portion indoors through the second air discharge port.
